(19) United States
(12) Patent Application Publication
(10) Pub. No.: US 2008/0220039 A1
(43) Pub. Date: Sep. 11, 2008

(54) Thin Film Medical Devices Manufactured on Application Specific Core Shapes

(76) Inventor: Darren R. Sherman, Fort Lauderdale, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/662,813

(22) PCT Filed: Sep. 16, 2005
(86) PCT No.: PCT/US05/33197

§ 371 Date: Mar. 14, 2007

Related U.S. Application Data
(60) Provisional application No. 60/610,778, filed on Sep. 17, 2004.

(51) Int. Cl. A61F 2/02; B32B 3/00

(52) U.S. Cl. 424/423; 427/2.24; 428/156

(57) ABSTRACT

A method for creating three-dimensional, unitary, thin film medical devices for implantation within a human subject is provided, along with a method for creating pores within such thin film devices. Using known sputtering methods, a film material is implanted on a core or combination of cores having an advanced threedimensional geometry, then the core is removed from the finished thin film device. The core may be provided with raised features at portions which are to be removed from the thin film device. Once the film has formed on the core, the portions of the film overlying the raised portions may be removed using mechanical means, such as grinding. Additionally, a kit can be provided having a plurality of the described thin film devices which may be used together for advanced surgical procedures.

### CROSS REFERENCE TO RELATED APPLICATION

This application claims priority from provisional patent application Ser. No. 60/610,778, filed Sep. 17, 2004, which is hereby incorporated herein by reference.

### FIELD OF THE INVENTION

This invention generally relates to a method for manufacturing three-dimensional, unitary medical devices implantable within a human subject and to medical devices of these types.

### DESCRIPTION OF RELATED ART

Medical devices that can benefit from the present invention include those that are characterized by hollow interiors and maneuverability. These include devices that move between collapsed and expanded conditions for ease of deployment through catheters and introducers. There is special application for medical devices which have porosity features, particularly porous walls. Examples include grafts, stents, occlusion devices, and medical devices which combine features from these types of devices. The present disclosure focuses upon occlusion devices for aneurysms or other defects or diseased locations within the body, explicitly including those that are sized, shaped and constructed for neural vascular use. Hereafter reference is made to occlusion devices although it is to be understood that the invention also finds application in other devices that are suitably made by the approaches and with the structures described herein.

In connection with the application of this invention to occlusion devices, these are typically for use in treating aneurysms. An aneurysm is an abnormal bulge or ballooning of the wall of a blood vessel. Typically, an aneurysm develops in a weakened wall of an arterial blood vessel. The force of the blood pressure against the weakened wall causes the wall to abnormally bulge or balloon outwardly. One detrimental effect of an aneurysm is that the aneurysm may apply undesired pressure to tissue surrounding the blood vessel. This pressure can be extremely problematic, especially in the case of an intracranial aneurysm where the aneurysm can apply pressure against sensitive brain tissue. Additionally, there is also the possibility that the aneurysm may rupture or burst, leading to more serious medical complications including mortality.

When a patient is diagnosed with an unruptured aneurysm, the aneurysm is treated in an attempt to reduce or lessen the bulging and to prevent the aneurysm from rupturing. Unruptured aneurysms have traditionally been treated by what is commonly known in the art as “clipping.” Clipping requires an invasive surgical procedure wherein the surgeon makes incisions into the patient's body to access the blood vessel containing an aneurysm. Once the surgeon has accessed the aneurysm, he or she places a clip around the neck of the aneurysm to block the flow of blood into the aneurysm which prevents the aneurysm from rupturing. While clipping may be an acceptable treatment for some aneurysms, there is a considerable amount of risk involved with employing the clipping procedure to treat intracranial aneurysms because such procedures require open brain surgery.

More recently, intravascular catheter techniques have been used to treat intracranial aneurysms because such techniques do not require cranial or skull incisions, i.e., these techniques do not require open brain surgery. Typically, these techniques involve using a catheter to deliver embolic devices to a preselected location within the vasculature of a patient. For example, in the case of an intracranial aneurysm, methods and procedures, which are well known in the art, are used for inserting and guiding the distal end of a delivery catheter into the vasculature of a patient to the site of the intracranial aneurysm. A vascular occlusion device is then attached to the end of a pusher member which pushes the occlusion device through the catheter and out of the distal end of the catheter where the occlusion device is delivered into the aneurysm.

Once the occlusion device has been deployed within the aneurysm, the blood clots on the occlusion device and forms a thrombus. The thrombus forms an occlusion which seals off the aneurysm, preventing further ballooning or rupture. The deployment procedure is repeated until the desired number of occlusion devices are deployed within the aneurysm. Typically, it is desired to deploy enough coils to obtain a packing density of about 20% or more, preferably about 35% and more if possible.

The most common vascular occlusion device is an embolic coil. Embolic coils are typically constructed from a metal wire which has been wound into a helical shape. One of the drawbacks of embolic coils for some applications is that they do not provide a large surface area for blood to clot thereto. Additionally, the embolic coil may be situated in such a way that there are relatively considerable gaps between adjacent coils in which blood may freely flow. The addition of extra coils into the aneurysm does not always solve this problem because deploying too many coils into the aneurysm may lead to an undesired rupture.

Therefore, there remains a need that is recognized and addressed according to the present invention for an occlusion device which provides a greater variation in options available to enhance the effectiveness of occupying the space within the aneurysm, including between adjacent occlusion devices, without increasing the risk of rupturing the aneurysm. Increasing surface area occupied by the device is also addressed by the invention to better promote clotting of blood.

Devices according to the invention typically fall under the category of thin film devices. Current methods of fabricating thin films (on the order of several microns thick) employ material deposition techniques. One example of a known thin film vapor deposition process can be found in Banas and Palmaz U.S. Patent Application Publication No. 2005/0033418, which is hereby incorporated herein by reference. Such methods attract the material of interest to geometrically simple core shapes until the desired amount has built up. The tendency to start with and keep these basic shapes (most commonly cylindrical primitives) would be driven by limitations of the apparatus and consistency of the field and material flow.

Traditionally, thin film is generated in a simple (oftentimes cylindrical, conical, or hemispherical) form and heat-shaped to create the desired geometry. However, in clinical applications there are instances where a shape (other than a cylinder and its heat-shaped derivatives) would be advantageous or would even facilitate a new treatment. Furthermore, manually constructing the desired shape out of cylindrical parts can be technically difficult and expensive.

Methods for manufacturing three-dimensional medical devices using planar films have been suggested, as in U.S. Pat. No. 6,746,890 (Gupta et al.), which is hereby incorporated herein by reference. However, the method described in Gupta et al. requires multiple layers of film material interspersed with sacrificial material. Accordingly, the methods described therein are time-consuming and complicated because of the need to alternate between film and sacrificial layers. Further, the devices described therein are ultimately created by inserting a core to separate two film layers, so it will be appreciated that there are significant limits on the geometry of the devices produced.

For some implantable medical devices, it is preferable to use a porous structure. Typically, the pores are added by masking or etching techniques or laser or water jet cutting. When occlusion devices are porous, especially for intracranial use, the pores are extremely small and these types of methods are not always satisfactory and can generate accuracy issues. Approaches such as those proposed by U.S. Patent Application Publication No. 2003/0018381, which is hereby incorporated herein by reference, include vacuum deposition of metals onto a deposition substrate which can include complex geometrical configurations. Microperforations are mentioned for providing geometric distendability and endothelization. Such microperforations are said to be made by masking and etching. Mandrels that receive the deposition can be patterned with a negative pattern, a positive pattern or a combination thereof. Also mentioned is that portions of the metallic layer not intended to be part of the deposited layer can be removed by machining, etching, laser cutting and the like. Another example of porosity in implantable devices is Boyle, Marton and Banas U.S. Patent Application Publication No. 2004/0098094, which is hereby incorporated by reference hereinto. This publication proposes endoluminal grafts having a pattern of openings, and indicates different orientations thereof could be practiced. These processes are said to be suitable for making stents or grafts, typically of an uncomplicated geometric shape.

Accordingly, a general aspect or object of the present invention is to provide a method for creating a three-dimensional, unitary implantable medical device which need not be cylindrical.

Another aspect or object of the invention is to provide a method for creating a three-dimensional implantable medical device using a continuous thin film.

Another aspect or object of the invention is to provide a method for creating an implantable device that need not be cylindrical from a three-dimensional thin film formed using known vapor deposition techniques.

Another aspect or object of the invention is to provide a method for creating pores in an implantable medical device formed on a core or mandrel.

Other aspects, objects and advantages of the present invention, including the various features used in various combinations, will be understood from the following description according to preferred embodiments of the present invention, taken in conjunction with the drawings in which certain specific features are shown.

### SUMMARY OF THE INVENTION

In accordance with the present invention, a method allows for the manufacture of medical devices, including ones that are geometrically advanced implantable medical devices of a unitary construction. When the terms “geometrically advanced” or “advanced three-dimensional geometry” are used herein, they are intended to refer to a three-dimensional shape which is not only a cylinder or a simple cylindrical derivative (e.g. a cone or toroid) or a hemisphere. A geometrically advanced core or mandrel is provided which is suited for creating a thin film by a physical vapor deposition technique, such as sputtering. A film material is deposited onto the core to form a seemless or continuous three-dimensional layer. The core then is removed by chemically dissolving the core, or by other known methods. In contrast to known methods, which involve joining cylindrical parts or planar films, the unitary part is less costly and less prone to mechanical failures.

The thickness of the thin film layer depends on the film material selected, the intended use of the device, the support structure, and other factors. A typical thin film layer of nitinol can be between about 0.1 and 250 microns thick and typically between about 1 and 30 microns thick. The thickness of the thin film layer can be between about 1 to 10 microns or at least about 0.1 microns but less than about 5 microns. Self-supporting ones can be thinner than supported ones.

The core may take any number of shapes, which allows for unitary devices with complicated features such as (but not limited to) stepped diameters, flares, bends, funnels, tapers, protrusions, indents, and the like. Furthermore, a plurality of sub-cores may be combined to create a single, unitary thin film device. Thus, the shape of the core (or sub-cores) can allow for complex geometries that do not require post-processing (e.g. assembling, laser cutting pores, placing on shaping mandrels, and heating) in order to create the desired part. Of course, post-processing procedures may be carried out without departing from the scope of the invention and may be desired in order to modify the performance characteristics of the final part. For example, traditional grinding and machining steps can be used to further develop the patterns (e.g. funnels, multiple stepped diameters, complex pore shapes, spheres, etc.) in the part.

Different anatomical locations can be treated with matching core shapes. By way of example, the core shapes for endoluminal stents will nearly always be generally tubular, but the present invention allows for countless variations, such as varying diameter, curvature, and branching configurations. Other exemplary features of complex core shapes include: single lumen bends (allowing for various diameters and radii of curvature); bifurcations (allowing for various diameters of the parent and branch vessels, various angles of incidence, and curvatures of the branch); T-joints (known to be useful for treating basilar tip aneurysms); plenums (allowing for various internal volume sizes, number of branches, diameters of branches); etc.

The core shapes and their complementary apparatus (mandrel, core or armature) can be constructed for the most difficult or the most common medical procedures where the part is applied. This allows the physician to perform a treatment with a device that fits the anatomy more closely. Depending on the degree of development that the deposition technology reaches, shapes could be fabricated that are treatment specific, anatomy specific, or even patient specific.

While core shapes suitable for routine treatments can be pre-fabricated and made readily available, it is contemplated that the parts manufactured according to the present invention may be combined for more complicated, less routine needs, procedures or operations. For example, core shapes for a complete set of modular components could be collected into a kit or “toolbox” of thin film mesh medical devices that the physician has at his or her disposal. When a physician or surgeon is presented with a case where treatment with a mesh is desired, the intended vessel areas could be covered with one or more meshes of appropriate size and shape for the anatomy at hand. This modular collection could be similar to the standard variety of piping components that are available for constructing fluid systems.

In addition, the core shape can also dictate the method of manufacture. For some implantable devices, such as occlusion devices, it is desirable to provide a porous structure. A mandrel or core that has raised features will create a part that mimics these raised features. The film overlying these raised features can then be removed using mechanical means such as grinding, machining, etching, cutting, or the like. Once removed, the remaining part will exclude the removed features and thus negate the need for laser cutting or etching as the primary tool. In this way, a mesh can be created that has openings shaped from the core mandrel, while the mesh is still on the mandrel. If a self-expanding film material such as nitinol is used, then expansion or contraction of the primary shape can then utilize these pores similar to existing self-expanding devices.

Special application for the present invention has been found for creating porous occlusion devices which cannot be formed using cylindrical parts or planar films. However, it will be seen that the method described herein is not limited to particular medical devices or particular surgical applications.

### BRIEF DESCRIPTION OF THE DRAWINGS

FIG. 1A is a schematic perspective view of a vascular area having a basilar tip aneurysm;

FIG. 1B is a schematic perspective view of the area shown in FIG. 1 with an aneurysm-occluding T-joint occlusion device according to an aspect of the present invention;

FIG. 1C is a perspective view of a mandrel or core used to form a T-joint device as generally shown in FIG. 1B;

FIG. 2 is a perspective view of a mandrel or core having raised features according to one aspect of the present invention;

FIG. 2A is a front elevational view of the mandrel or core of FIG. 2;

FIG. 2B is a side elevational view of the mandrel or core of FIG. 2;

FIG. 2C is a top plan view of the mandrel or core of FIG. 2;

FIG. 3 is a perspective view of a thin film device created using the core of FIG. 2;

FIG. 4 is a perspective view of a combination core according to an aspect of the present invention;

FIG. 5 is a perspective view of an embodiment of a mandrel or core according to an aspect of the present invention;

FIG. 5A is a front elevational view of the mandrel or core of FIG. 5;

FIG. 5B is a side elevational view of the mandrel or core of FIG. 5;

FIG. 5C is a top plan view of the mandrel or core of FIG. 5;

FIG. 6 is a perspective view of an embodiment of a mandrel or core according to an aspect of the present invention;

FIG. 7 is a perspective view of an embodiment of a mandrel or core according to an aspect of the present invention;

FIG. 7A is a front elevational view of the mandrel or core of FIG. 7;

FIG. 8 is a perspective view of an embodiment of a mandrel or core according to an aspect of the present invention;

FIG. 9 is a perspective view of an embodiment of a mandrel or core according to an aspect of the present invention;

FIG. 9A is a front elevational view of the mandrel or core of FIG. 9;

FIG. 9B is a side elevational view of the mandrel or core of FIG. 9;

FIG. 9C is a top plan view of the mandrel or core of FIG. 9;

FIG. 10 is a perspective view of an embodiment of a mandrel or core according to an aspect of the present invention;

FIG. 10A is a side elevational view of the mandrel or core of FIG. 10;

FIG. 11 is a perspective view of an embodiment of a mandrel or core according to an aspect of the present invention;

FIG. 11A is a side elevational view of the mandrel or core of FIG. 11;

FIG. 12 is a perspective view of an embodiment of a mandrel or core according to an aspect of the present invention;

FIG. 13 is a perspective view of an embodiment of a mandrel or core according to an aspect of the present invention;

FIG. 13A is a front elevational view of the mandrel or core of FIG. 13;

FIG. 14 is a perspective view of an embodiment of a mandrel or core according to an aspect of the present invention;

FIG. 14A is a front elevational view of the mandrel or core of FIG. 14;

FIG. 14B is a side elevational view of the mandrel or core of FIG. 14;

FIG. 14C is a bottom plan view of the mandrel or core of FIG. 14;

FIG. 15 is a perspective view of an embodiment of a mandrel or core according to an aspect of the present invention;

FIG. 15A is a front elevational view of the mandrel or core of FIG. 15; and

FIG. 15B is a top plan view of the mandrel or core of FIG. 15.

### DESCRIPTION OF THE PREFERRED EMBODIMENTS

As required, detailed embodiments of the present invention are disclosed herein; however, it is to be understood that the disclosed embodiments are merely exemplary of the invention, which may be embodied in various forms. Therefore, specific details disclosed herein are not to be interpreted as limiting, but merely as a basis for the claims and as a representative basis for teaching one skilled in the art to variously employ the present invention and virtually any appropriate manner.

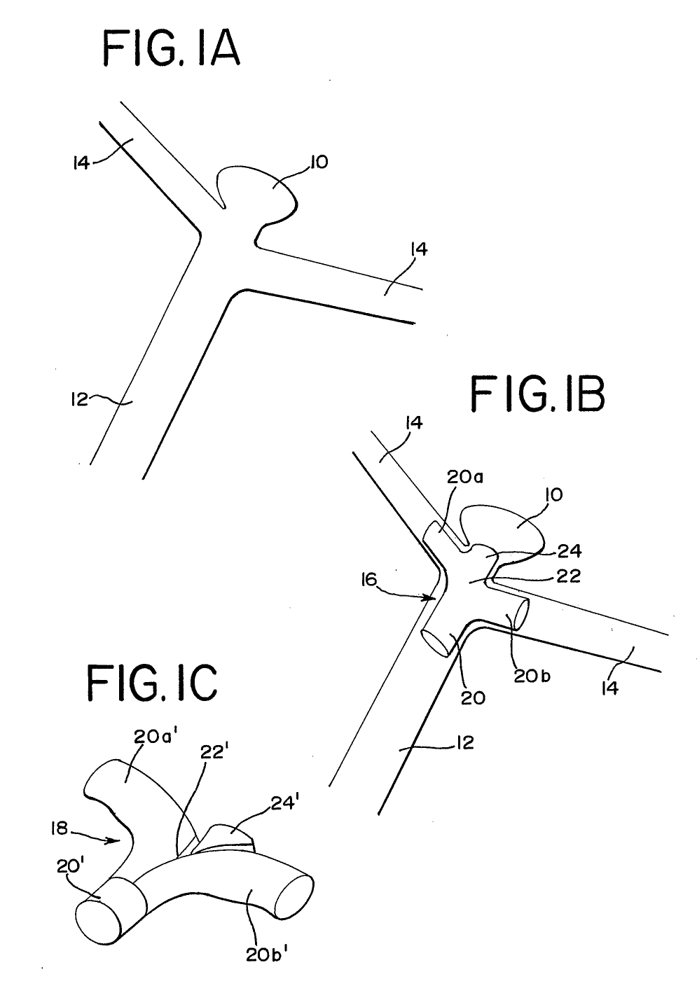

FIG. 1A illustrates a basilar tip aneurysm 10, although it is also indicative of other aneurysms at branching locations of blood vessels (“bifurcation aneurysms”). In treating a basilar tip aneurysm 10, blood must be prevented, or at least significantly restricted, from entering the aneurysm 10, without preventing blood flow through the parent or main vessel 12 and branch vessels 14. The aneurysm-occluding T-joint occlusion device illustrated in FIG. 1B, generally designated as 16, is specially adapted to treat bifurcation aneurysms as illustrated in FIG. 1A. The device 16 has a three-dimensional, unitary thin film construction formed according to the present invention. As discussed in further detail herein, the device 16 is preferably formed of an expandable film material such as nitinol. FIG. 1C shows a core 18 suitable for making the FIG. 1B occlusion device 16.

It will be seen that the occlusion device 16 has three generally tubular legs 20, 20a and 20b joined at a central junction 22. Also joined to the junction 22 is an occlusion member 24, which is configured to be inserted at least partially into the aneurysm 10. Preferably, the occlusion member 24 is delivered to the aneurysm 10 in a contracted state. It is allowed to expand in order to better occlude or restrict blood flow into the aneurysm 10. Accordingly, it is preferable to use a shape memory alloy, such as nitinol which can be delivered in a contracted configuration that will allow the occlusion member 24 to fit in its contracted state within the mouth of the aneurysm 10, and later expand to the appropriate size when within the aneurysm 10.

When nitinol is used as the film material, it can be sputter-deposited onto the core 18 in either a martensitic state or an austenitic state. Nitinol applied to the core 18 in its martensitic state can be heat treated and shaped into the appropriate austenitic configuration. Alternatively, the sputtering conditions may be such that the nitinol film adheres to the core 18 in its austenitic state, which is later transferred to a temporary martensitic state for implantation within the aneurysm 10 and vessels 12 and 14. Preferably, the composition of the nitinol is such that it undergoes a phase shift from martensite to austenite at a temperature slightly below body temperature, which causes the occlusion device 16 to expand once it has been implanted within the body. The nitinol thin film may be created using known sputtering procedures and equipment, including either a cylindrical or flat sputtering source.

Additionally, the devices can be of a type that is not self-expanding. They can be expanded by any suitable expansion implement, such as a balloon cathether and the like. They are formed by deposition onto a core or mandrel as generally discussed herein, and the material will be expandable when subjected to expansion forces by a suitable medical device.

In order to create the occlusion device 16, a core 18 having sections 20′, 20a′, 20b′, 22′, and 24′ which correspond in position but not necessarily length to legs 20, 20a, 20b, junction 22, and occlusion member 24 respectively of the occlusion device 16 is provided. The core 18 is inserted into a sputtering chamber and one or more thin film layers of biocompatible material are sputtered onto its outer surface. The film material, in the general form of the occlusion device 16, is subsequently removed from the core 18 using known techniques.

When desired, the core 18 and its resulting occlusion device 16 can be uniquely tailored to match the aneurysm of the particular patient. For example, a three-dimensional representation of the aneurysm and surrounding blood vessels for a particular patient may be created using magnetic resonance imaging (MRI) or similar imaging techniques. Following this approach, the three-dimensional image is translated into a form readable by the apparatus used to create the core or mandrel. The core or mandrel then is created according to the three-dimensional image and a thin film is sputter-deposited onto the core. Thus, beyond providing a treatment-specific device (e.g. occlusion device 16 for a basilar tip aneurysm), the present invention allows for the creation of patient-specific devices.

The legs 20, 20a, and 20b of device 16 may be provided with pores or fenestrations, not illustrated, in order to allow for enhanced blood flow. The pores may be provided according to known methods, such as etching or laser cutting. In accordance with an embodiment, pores can be created using a core or mandrel with features that facilitate pore formation.

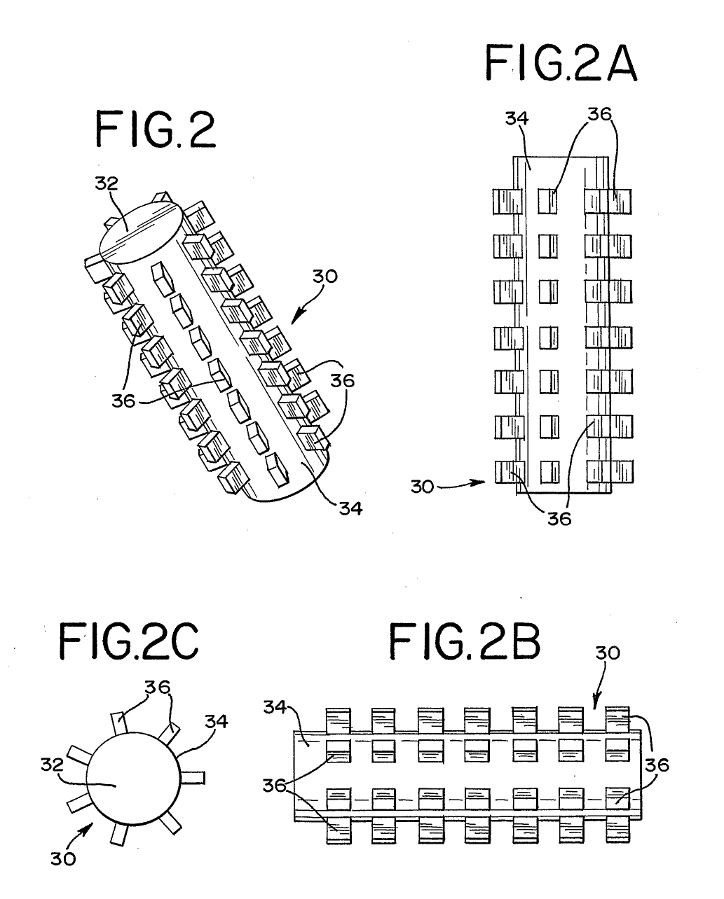

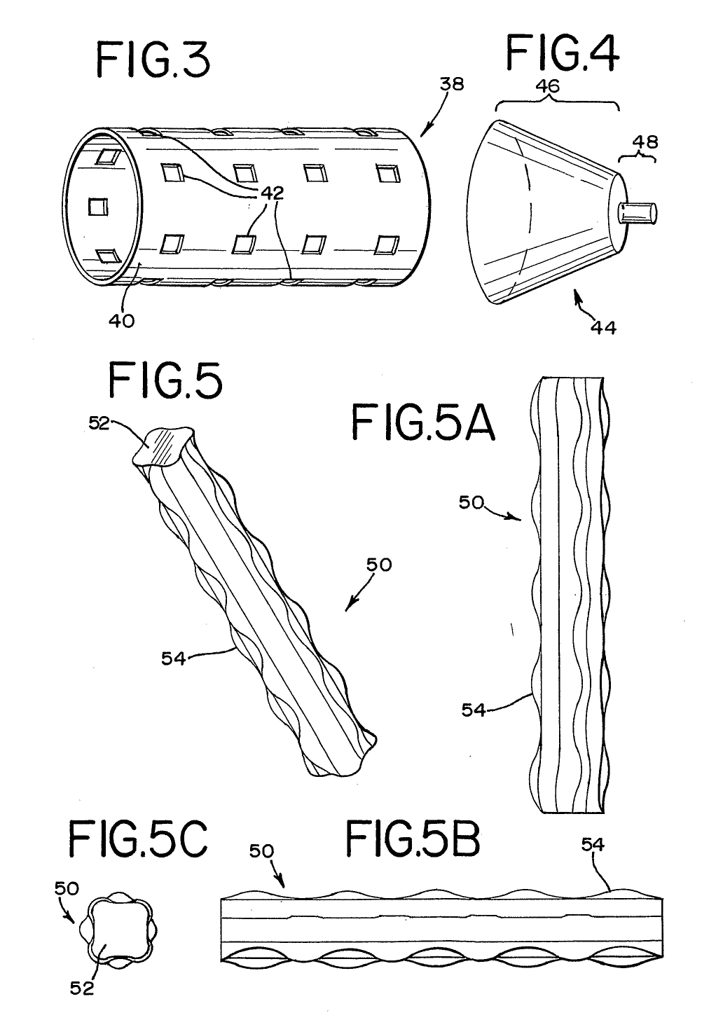

FIGS. 2, 2A, 2B and 2C illustrate a representative core 30 with a body 32 having a base surface 34 and a plurality of raised features 36. The term “base surface” is best understood with reference to the thin film occlusion device 38 ultimately created through such an approach, as shown in FIG. 3. Generally speaking, the raised features 36 of the core 30 are located where portions of the thin film are to be removed, while the base surface 34 of the core 30 provides the location for forming the portion of the thin film which is to remain for the implantable device (i.e. all portions of the thin film in closer proximity to a central axis of the core than the raised features).

A thin film is applied to the core 30 according to known deposition procedures such as sputtering, which will result in a thin film having projections (not illustrated) that mimic the underlying raised features 36 of the core 30. The projections are removed to form the pores 42. Such projections preferably are removed with mechanical means, such as grinding or milling. Once the projections have been removed, the thin film device 38 is characterized by the remaining film material 40 and the openings or pores or fenestrations 42. It can be said that the portions 40 of film which remain after grinding are disposed above the base surface 34 of the core 30 at this stage of preparation. While the core 30 shown in FIG. 2 is geometrically simple with identical raised features 36 in a regular pattern, it is contemplated that the present invention can be applied to any anatomically useful core shape with other raised features, with regularly or irregularly shaped raised features in any possible pattern of configuration along the base surface 34. Preferably, all of the raised features 36 extend an equal distance above the base surface 34, which simplifies the step of removing the projections from the thin film. With such approaches, complex pore configurations may be created without an attendant increase in the difficulty of cutting the openings or pores.

In general, the present invention may be practiced using any biocompatible material which is susceptible to sputter-deposition. While polymers could be suitable in the proper circumstances, metals are usually better suited to the types of devices and methods of the present invention. For example, platinum and tungsten may be optimally used for certain devices, as generally appreciated by those skilled in the art. Preferred are metal alloys, especially alloys including nickel and tungsten and the nitinol metals discussed herein.

According to another aspect of the present invention, a kit or toolbox is provided which includes a plurality of thin film devices having geometrically advanced configurations. Preferably, the kit has several devices according to the present invention with differing shapes and can also include known thin film devices, such as cylindrical or conical implants. While it is contemplated that the methods described herein can be used to produce extremely complex devices, it is also understood that it may be impractical in all situations to implant devices that are all of the same shape or characteristics. In such situations, a kit with a variety of implants allows the medical practioner to use different medical devices such as occlusion devices to be delivered to an aneurysm or the like so as to maximize the occlusion packing effect by choosing each device according to the volume of an aneurysm or portion of an aneurysm in need of occlusion.

Examples of varieties of occlusion device shapes which can be in accordance with the present invention are now discussed with reference to representative cores illustrated in FIGS. 5 through 15B. Different varieties can be provided for the aforementioned kit or toolbox approach. Variations can include those of shape, porosity, materials, size, relative angles, collapsibility, springability and so forth. It will be appreciated that different varieties can be suitable for respective particular situations, depending on the shape, size, condition and disease characteristics of the aneurysm being occluded, as well as upon the position and characteristics of other occlusion devices being used to treat the aneurysm or the like.

FIGS. 5, 5A, 5B and 5C illustrate a core 50 with an advanced three-dimensional geometry that can be described as a column having a generally square base 52 and bumpy or undulating sidewalls 54. The core 50 provides the template for the medical device being prepared, which can be followed by imparting porosity to the device.

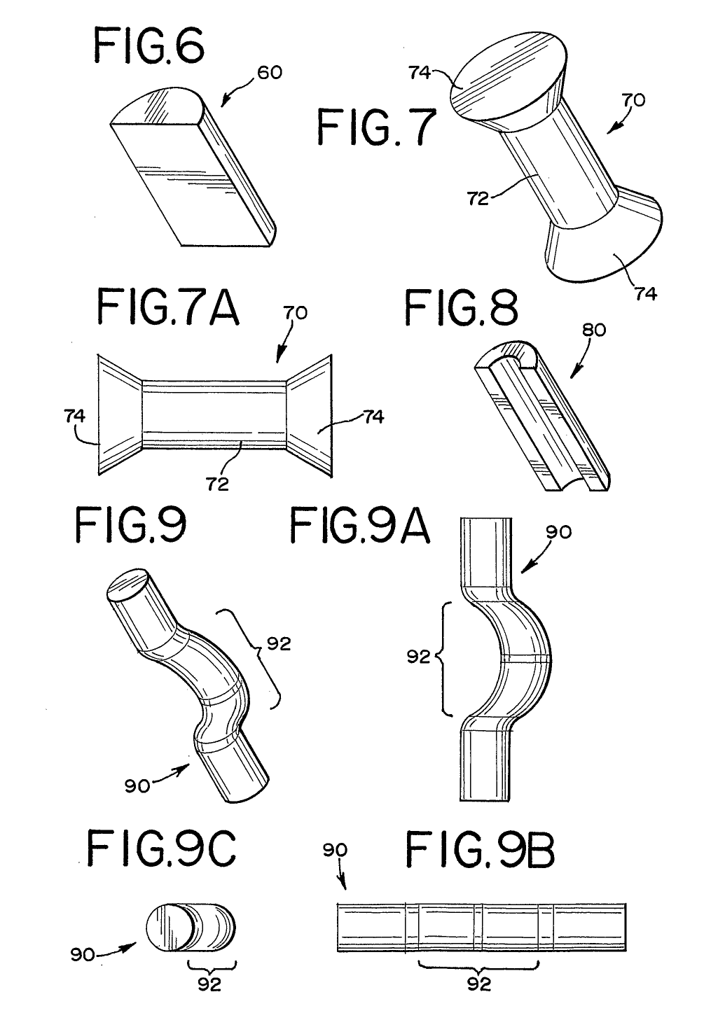

FIG. 6 illustrates a core 60 with an advanced three-dimensional geometry that can be described as generally “D-shaped.” This is for use in preparing a D-shaped thin film mesh that can be used in medical devices, especially occlusion devices.

FIGS. 7 and 7A illustrate a core 70 with an advanced three-dimensional geometry that includes a cylindrical midsection 72 and outwardly flared end caps 74. A thin film medical device prepared on this core 70 has a configuration useful in, for example, occluding locations having shapes of varying widths.

FIG. 8 illustrates a core 80 with an advanced three-dimensional geometry that can be described as a half-pipe. An occlusion device prepared on such a core 80 has good flexibility and can be used to sandwich into relatively thin volume locations of an aneurysm or the like.

FIGS. 9, 9A, 9B and 9C illustrate a core 90 with an advanced three-dimensional geometry that includes a generally uniform circular cross-section and a hump or curve or bend 92. A thin film device prepared from such a core 90 provides a configuration that can be manipulated into oddly shaped areas. With a porous thin film structure, endothelial growth thereinto typically is very advantageous.

FIGS. 10 and 10A illustrate a core 100 with an advanced three-dimensional geometry that includes a cylindrical section 102 abutting a toroidal section 104, which shape can be likened to a lollipop. The open area defined by the toroidal section 104 can be used to surround other devices or provide an opportunity for deformation of the toroidal shape for good packing characteristics.

FIGS. 11 and 11A illustrate a core 110 with an advanced three-dimensional geometry that includes a cylindrical section 112 with a plurality of rectangular bumps 114 spaced apart from each other in a row. After coating with thin film nitinol or other suitable material, the bumps 114 are machined off, together with the thin film thereon in order to thereby form pores in the thin film. When the core 110 is dissolved away, the result is a thin film cylinder having a row of generally rectangular pores in general alignment along a length of the resulting medical device.

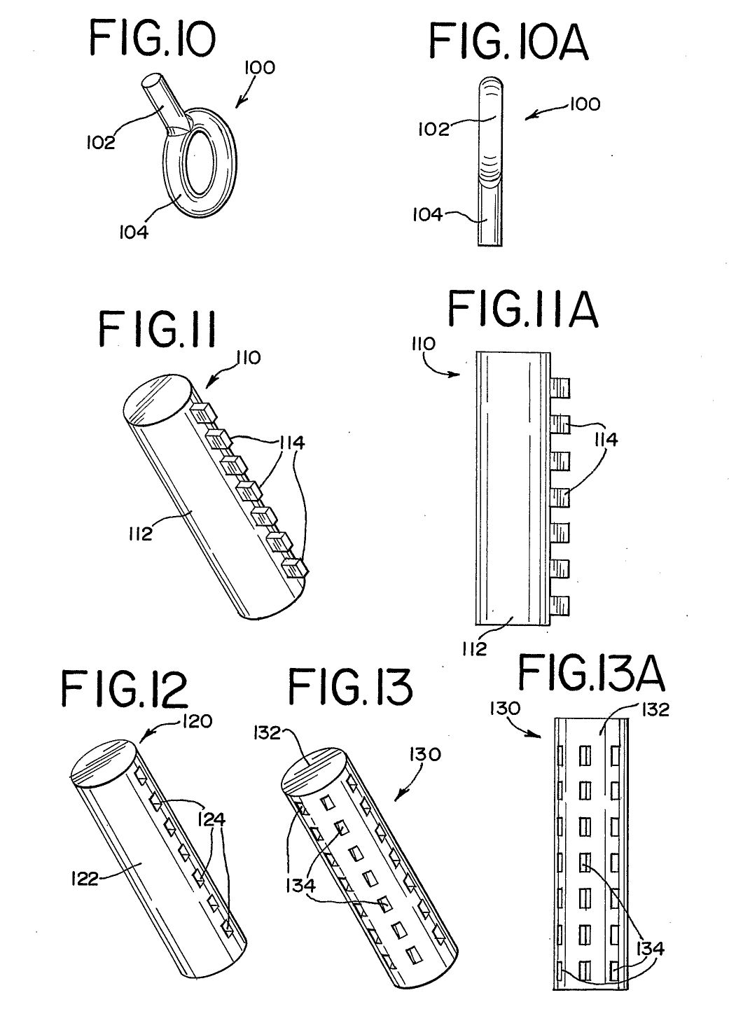

FIG. 12 illustrates a core 120 with an advanced three-dimensional geometry that includes a cylindrical section 122 with a plurality of rectangular holes 124 spaced apart from each other in a row. The holes 124 can be shallow or pass through the entirety of the cylindrical section 122 or may have varying depths.

FIGS. 13 and 13A illustrate a core 130 with an advanced three-dimensional geometry that includes a cylindrical section 132 with a plurality of rectangular holes 134 arranged in a uniform grid pattern.

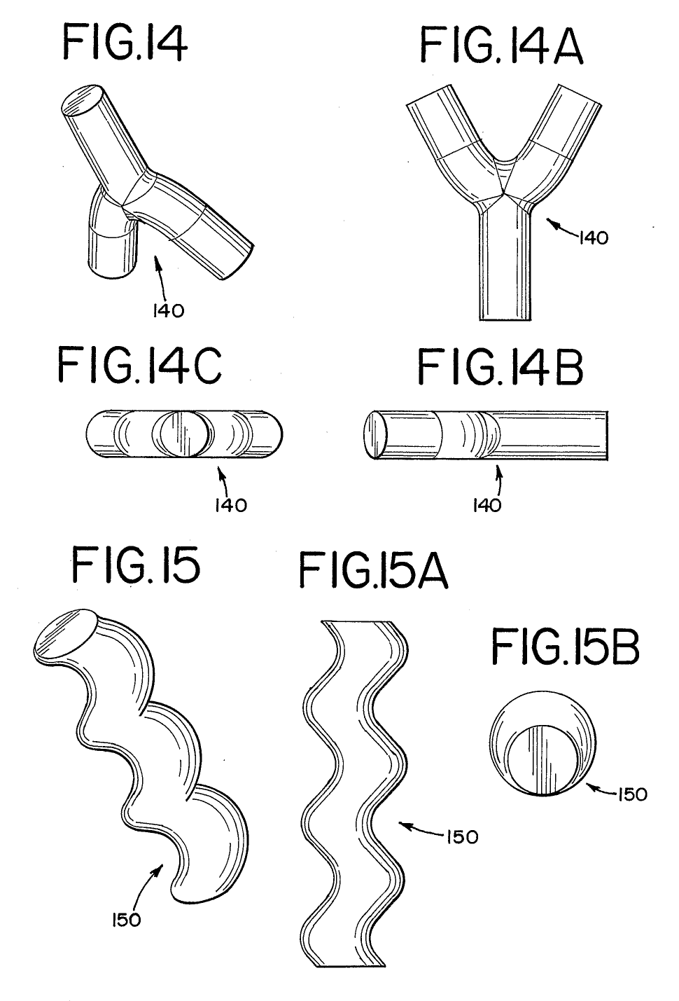

FIGS. 14, 14A, 14B and 14C illustrate a core 140 with an advanced three-dimensional geometry that can be described as a Y-joint. Such a core can be useful in making a Y-shaped occlusion device that has operational characteristics on the order of those of the device shown in FIG. 1B.

FIGS. 15, 15A and 15B illustrate a core 150 with an advanced three-dimensional geometry that can be described as a helix. The core 150 can also be understood as a variation on an embolic coil. The curved shaping given to occlusion devices or the like made with such a core 150 open up important possibilities for fitting into unusually shaped openings and/or for enhanced packing into an aneurysm.

Heretofore, the creation of geometrically advanced thin film occlusion devices has been described with reference to a mandrel or core having a single geometric shape. However, it is contemplated that a plurality of cores may be combined, stacked, or otherwise assembled together in order to provide a mandrel or core of more varied shapes.

For example, FIG. 4 shows a core 44 which can be formed either as an integral unit or as a combination core having two stacked sub-cores: a frusto-conical sub-core 46 and a cylindrical sub-core 48. The two sub-cores 46 and 48 can be joined by any means, provided that the thin film device created using the combination mandrel or core is continuous and unitary. Otherwise, the same principles are used in forming the biocompatible film material and importing porosity thereto as desired. By way of further example, the core 18 of FIG. 1C can be provided as a combination core formed by joining at junction section 22′ four sub-cores corresponding to sections 20′, 20a′, 20b′, and 24′.

It will be understood that the embodiments of the present invention which have been described are illustrative of some of the applications of the principles of the present invention. Numerous modifications may be made by those skilled in the art without departing from the true spirit and scope of the invention, including those combinations of features that are individually disclosed or claimed herein.

1. A method of creating a device suitable for implantation within a human subject, comprising:
providing a core with an advanced three-dimensional geometry;
disposing said core within an interior of a vapor deposition chamber;
depositing a continuous, unitary layer of a biocompatible film material over the core from a vapor source associated with the interior of the vapor deposition chamber; and
separating the layer of film material from the core.

2. The method of claim 1, wherein said separating includes removing said core from within said layer of film material by dissolving said core.

3. The method of claim 1, wherein said biocompatible film material is nitinol.

4. The method of claim 3, wherein said nitinol is a martensite thin film.

5. The method of claim 3, wherein said nitinol is an austenite thin film that transitions from martensite to austenite upon exposure to human body temperature.

6. The method of claim 1, wherein said biocompatible film material has a thickness greater than about 0.1 microns and less than about 5 microns.

7. A core for creating a device suitable for implantation within a human subject using a vapor deposition chamber comprising a body having a base surface interspersed with a plurality of raised features, wherein said raised features extend from the base surface.

8. The core of claim 7, said base surface having an advanced three-dimensional geometry.

9. A method of creating openings within a device suitable for implantation within a human subject, comprising:
providing a core having a base surface interspersed with a plurality of raised features;
disposing said core within an interior of a vapor deposition chamber;
depositing a continuous, unitary layer of a biocompatible film material over the core from a vapor source associated with the interior of the vapor deposition chamber, such that the layer includes a plurality of projections directly overlaying said plurality of raised features; and
removing the projections from the layer of film material.

10. The method of claim 9, wherein said separating includes removing said core from within said layer of film material by dissolving said core.

11. The method of claim 9, said base surface having an advanced three-dimensional geometry.

12. The method of claim 9, wherein the step of removing the projections includes grinding or milling.

13. The method of claim 9, wherein said biocompatible film material is nitinol.

14. The method of claim 13, wherein said nitinol is a martensite thin film.

15. The method of claim 13, wherein said nitinol is an austenite thin film that transitions from martensite to austenite upon exposure to human body temperature.

16. A surgical implant kit comprising a plurality of thin film devices suitable for implantation within a human subject, wherein at least one of said devices has an advanced three-dimensional geometry and at least two of said devices have a shape different from each other and from said advanced three-dimensional geometry.

17. A method of shaping a core for creating a device suitable for implantation within a human subject comprising:
creating a three-dimensional image of an implantation site within a human subject;
translating the three-dimensional image into a form of information readable by a core-shaping apparatus;
transferring said readable information to said core-shaping apparatus; and
operating said core-shaping apparatus to create a core shaped to generally conform to at least a portion of the three-dimensional image.

18. A method of creating a device suitable for implantation within a human subject, comprising:
providing a plurality of sub-cores, wherein each of said sub-cores defines a three-dimensional surface and is suitable for vapor deposition of a biocompatible thin film material on said surface;
joining said plurality of sub-cores to form a combination core;
disposing said combination core within an interior of a vapor deposition chamber;
depositing a continuous, unitary layer of a biocompatible film material over the combination core from a vapor source associated with the interior of the vapor deposition chamber; and
separating the layer of film material from the combination core.

19. The method of claim 18, wherein said separating includes removing said core from within said layer of film material by dissolving said core.

20. The method of claim 18, said combination core having an advanced three-dimensional geometry.

21. The method of claim 20, wherein said joining combines sub-cores which have different geometric configurations.

22. The method of claim 18, wherein said biocompatible film material is nitinol.

23. The method of claim 22, wherein said nitinol is a martensite thin film.

24. The method of claim 22, wherein said nitinol is an austenite thin film that transitions from martensite to austenite upon exposure to human body temperature.
